(19) United States
(12) Patent Application Publication
(10) Pub. No.: US 2007/0032774 A1
(43) Pub. Date: Feb. 8, 2007

(54) Container for transporting blood and blood products

(76) Inventors: Clifford Glade, Islamorada, FL (US); Kenneth Wagner, Islamorada, FL (US); Jim Leclair, Covington, GA (US); Randal Miller, Peachtree city, GA (US); Ray Sheppard, Cooper City, FL (US)

Correspondence Address: AKERMAN SENTERFITT, P.O. BOX 3188, WEST PALM BEACH, FL, 33402-3188, US

(21) Appl. No.: 11/333,194

(22) Filed: Jan. 17, 2006

Related U.S. Application Data
(63) Continuation-in-part of application No. 11/281,548, filed on Nov. 17, 2005.
(60) Provisional application No. 60/681,769, filed on May 17, 2005.
(60) Provisional application No. 60/681,774, filed on May 17, 2005.
(60) Provisional application No. 60/681,770, filed on May 17, 2005.
(60) Provisional application No. 60/682,318, filed on May 18, 2005.

(51) Int. Cl. A61B 19/00

(52) U.S. Cl. 604/403

(57) ABSTRACT

A reusable container for transporting blood and blood products, which includes an outer case including a lid, and an insulating layer substantially inside the outer case and the lid. A generally rigid water-resistant well has a lip which abuts walls of the outer case so as to substantially seal the insulated layer to the outer case. At least one cooling element is provided, which is capable of maintaining temperature within a prescribed temperature range for predetermined periods of time, the cooling element being disposed within the well and at least partially surrounding a receptacle area. A caddy is located in the receptacle area, at least partially surrounded by the cooling element, and is designed to accommodate at least one unit of blood or blood products. The cooling element may include phase change gel sealed within a plastic surround, and is designed to be separately removable and freezable prior to use.

### CROSS-REFERENCE TO RELATED APPLICATIONS

The present application is a Continuation-In-Part to U.S. patent application Ser. No. 11/281,548 filed Nov. 17, 2005 which claims priority to U.S. Provisional Patent Application No. 60/681,769 filed May 17, 2005. The present application also claims priority to U.S. Provisional Patent Application No. 60/681,774 filed May 17, 2005, U.S. Provisional Patent Application No. 60/681,770 filed May 17, 2005 and U.S. Provisional Patent Application No. 60/682,318 filed May 18, 2005. The entire disclosures of these earlier applications are incorporated herein by reference.

### FIELD OF THE INVENTION

The invention relates to an improved container for transporting blood and blood products at the required temperature range for extended periods of time without the use of either wet or dry ice, or gel packs, in a durable, reusable container that is capable of maintaining and monitoring the temperature in transit for compliance with certain protocols.

### BACKGROUND OF THE INVENTION

Transporting blood and blood products in a timely manner has always been a problem. Generally, the health care industry has relied on the use of insulated containers such as coolers developed and manufactured for the recreational industry for keeping food and beverages cold. However, the coolers that have been used do not address the need for documented temperature monitoring for blood products for transfusions that the Food and Drug Administration (FDA) requires as of 2004. Thus, the healthcare industry is in need of a better solution for the transportation of blood, blood products, human tissues, stem cells, and donor organs, which require documented temperature tracking for FDA compliance.

Spoilage and the risk of infection from stale blood, blood products, tissues, cells or donor organs are also of paramount concern. The FDA requires that blood and blood products being transported must be maintained at a certain temperature range. For instance, packed red blood cells should be maintained at a temperature range of 1° C. to 6° C.; fresh plasma thawed (for use) requires a temperature range of 1° C. to 6° C.; platelet concentrates must be maintained at 20° C. to 24° C.; and fresh frozen plasma must be stored at or below −25° C.

Additionally, transporting blood and blood products requires that certain protocols be met in order to prevent exposure, damage or contamination while in transit. Many blood banks try to solve this problem by transporting blood and blood products in a leak-proof primary container placed in a secondary container, such that the specimen is protected from inadvertent puncture or contamination. The units of red blood cells are then packed on wet or dry ice or a chemical coolant to be transported in Styrofoam® containers.

However, one of the limitations of this method of transportation is that wet ice is messy, often spills during transportation, and re-icing while in transit is sometimes challenging in remote areas. Dry ice offers its own complications as it can be hazardous as, for example, loose dry ice can inadvertently touch the blood or blood product, causing it to freeze the blood or blood product at the area of contact and damage the entire unit. Additionally, dry ice is sometimes unavailable in certain parts of the world. These factors only serve to further limit the ability to transport blood and blood products for prolonged periods. To alleviate this problem, frozen gel packs have been used to help maintain required temperatures. However, gel packs are susceptible to leaks and can therefore be unreliable. Additionally, it is difficult to maintain a consistent temperature across the whole unit of blood or blood product with gel packs.

Another disadvantage is that the blood and blood products have limited viability over prolonged periods even if transported at the required temperature range. Red blood cells when stored at the required temperature range are viable for up to six (6) hours, while blood platelets are limited to four hours expiration time. Conventional insulated shipping containers do not address this problem, which can be further complicated when blood or blood products are being transported internationally. Thus, there still exists a need to be able to transport blood and blood products efficiently and safely for periods lasting longer than four to six hours without the use of either wet or dry ice, or gel packs in a durable, reusable container that is capable of maintaining or monitoring the temperature in transit for compliance with certain protocols.

The transportation of platelet concentrates is particularly problematic, because of the need to keep such concentrates within a temperature range of between 20° C. and 24° C. Such temperature ranges are very difficult to maintain using dry and wet ice, which tend to cool the product too much. The current solutions to the transportation of platelet products include packing the containers with gel packs that are warmed to room temperature, and surrounding the products with several layers of insulation. The resulting packages are bulky, and there is also no way to ensure that the temperature stays constant through different weather conditions (and varying ambient temperatures in hospitals and laboratories) that the package may be transported through. Platelet concentrates are typically transported in lots of 5, with 5, 10, 15 or 20 as the usual shipment.

### SUMMARY OF THE INVENTION

The invention is directed to a reusable container for transporting blood and blood products providing the ability to not only transport, but also to monitor and maintain the refrigerated blood or blood products at the necessary temperature range for more than (for example) twenty-four hours without the use of wet or dry ice, or gel packs. The container is capable of transporting 1 to 2, 1 to 4, or 1-6 or more units of blood from the blood bank to Operating Room, Emergency Room, dialysis, cancer center, home health care facilities or any other facility where blood is used.

In one arrangement, the container comprises an outer case including a lid, an insulating layer substantially inside the outer case and the lid, and a generally rigid water-resistant well having a lip, wherein the lip abuts walls of the outer case so as to substantially seal the insulated layer to the outer case. A least one cooling element is included, the at least one cooling element being capable of maintaining temperature within a prescribed temperature range for predetermined periods of time upon being kept at a predetermined temperature prior to use, the at least one cooling element being disposed within the well and at least partially surrounding a receptacle area. A caddy is located in the receptacle area and designed to accommodate at least one unit of blood or blood products, wherein the caddy is at least partially surrounded by the at least one cooling element.

In another arrangement, a container for transporting blood and blood products comprises an outer case including a lid, an insulating layer substantially inside the outer case and the lid, and at least two cooling elements, the cooling elements each being capable of maintaining temperature within a prescribed temperature range for predetermined periods of time upon being kept at a predetermined temperature prior to use, the cooling elements being disposed within the insulating layer and each defining a receptacle area designed to accommodate at least one unit of blood or blood products. Each cooling element is designed to maintain blood or blood products located within the receptacle area at a different predetermined temperature.

In a further arrangement, a container for transporting blood and blood products includes an outer case including a lid, an insulating layer substantially inside the outer case and the lid, and a flexible foil barrier positioned between the outer case and the insulating layer. At least one cooling element is included, the at least one cooling element being capable of maintaining temperature within a prescribed temperature range for predetermined periods of time upon being kept at a predetermined temperature prior to use, the at least one cooling element being disposed within the flexible foil barrier and at least partially surrounding a receptacle area. A receptacle is located in said receptacle area and designed to accommodate at least one unit of blood or blood products, wherein the receptacle is at least partially surrounded by the at least one cooling element.

The container can include a waterproof outer case, which may be constructed from hard, durable, rustproof material or a soft, flexible, lightweight material that is durable and stain resistant. The outer case provides for varied means for lifting and towing the container, which may be singularly employed or in combination thereof, for example a reinforced handle on the top of the container, wheels, a telescopic handle, non-retractable handles and shoulder straps.

The exterior of the container can contain at least one information sleeve adapted to hold identification information concerning the blood or blood product being transported. A patient's name, social security or identification number, surgeon, blood type, date the blood or blood product left the blood bank, scheduled date of delivery, number of units of blood, delivery operating room number, surgeon, doctor and medical procedure can be documented and stored in the information sleeve.

The cooling element is an important feature of the container in accordance with the present invention. The cooling element can include four modular units shaped and sized to securely fit the top, bottom and sides of the inner perimeter of the well and are adapted to form a receptacle area for housing an inner caddy. Alternatively, the cooling element can be in one piece and can include a bottom, side walls and at least one top portion, wherein the top portion is hingedly attached to the side walls. The cooling element is responsible for keeping the inner caddy at the required temperature (for example, 1° C. to 6° C.), and is made from strong, durable and lightweight material, such as ABS plastic. A cooling material is disposed in all or part of the cooling elements and may include a phase change gel. The cooling element is adapted to, when frozen and conditioned, maintain the required temperature for extended periods of time, i.e. 24+ hours. The cooling elements can be frozen prior to each day's use in any standard or commercial freezer for up to 24 hours prior to each use. The cooling elements are stackable and provide for easy storage in a freezer. After removal from the freezer, the cooling elements can be conditioned prior to use and insertion into the container. The object of conditioning the cooling elements is to allow it to transition from the temperature of the freezer to a temperature proximal to the desired phase change temperature. This can be achieved simply by exposing the cooling elements to the ambient room temperature by placing it out on a counter (not in the insulated case), for approximately 30 to 60 minutes.

The container can include an inner caddy, which provides for a bottom portion connected to sidewalls, a front wall, a back wall and a middle divider. Lifting means can be provided for lifting and removing the inner caddy from the container. Lifting means may include a handle, strap, tab, or anything that can be grasped or is generally known and used by those skilled in the arts. In one arrangement, the walls include one or more top portions that are hingedly connected to a lid extending outwardly open from the receptacle area to form gull wings. In another arrangement, the inner caddy opens from front to back providing access to its contents. The inner caddy is removable with cleaning vents, allowing for self-drainage and ease of cleaning. The inner caddy is conveniently sized such that it may be stored in a standard or commercial refrigerator until the blood or blood product is ready for use.

The container can be further equipped with a receiving area for a temperature data-logging device which comprises of at least one probe for monitoring, collecting and reporting data concerning the blood or blood units being transported. Suitable temperature data-logging devices may include for example a Validator, which consists of proprietary technology currently available through American Thermal Wizard International, Inc. The temperature data-logging device can be programmable to meet the compliance requirements and will continuously monitor the temperature of the blood or blood products while it is housed in the container. The information once logged can be graphed or logged and is capable of being downloaded into Microsoft Word® or Excel®. The temperature data-logging device can be programmed to either transmit an alarm or a page if the temperature range falls or exceeds the set parameters.

The container can be further equipped with a GPS device and/or a cell phone so that the position of the container can be tracked, and optionally reported to a user in real time.

For a further and more fully detailed understanding of the present invention, various objects and advantages thereof, reference is made to the following detailed description and the accompanying drawings.

### BRIEF DESCRIPTION OF THE DRAWINGS

Further objectives and advantages of the invention will become more apparent from the following description and claims and from the accompanying drawings herein.

FIG. 1 depicts a container for transporting blood and blood products according to the present invention.

FIG. 2 depicts an external view of one arrangement of the container for transporting blood and blood products.

FIG. 3 depicts an alternate arrangement of the container for transporting blood and blood products.

FIG. 4a depicts an arrangement of the cooling elements of the invention.

FIG. 4b depicts an arrangement of the cooling elements of the invention.

FIG. 5a depicts one arrangement of the inner caddy.

FIG. 5b depicts another arrangement of the inner caddy.

FIG. 6 depicts an alternate arrangement of the container according to the invention for transporting vials of specimens.

FIG. 7a depicts an alternate arrangement of the container according to the invention.

FIG. 7b depicts an alternate arrangement of the container according to the invention.

FIG. 8 depicts an alternate arrangement of the container according to the invention.

### DESCRIPTION OF PREFERRED EMBODIMENTS

The terms “blood and blood products” as used herein are not intended to be limited terms, but are used in an exemplary, non-limiting manner. Wherever the terms “blood and blood products” are used, it should be appreciated that any type of human or animal blood, cells including stem cells, bone marrow, donor organs, tissue products, plasma concentrates, specimens and the like are intended to be covered.

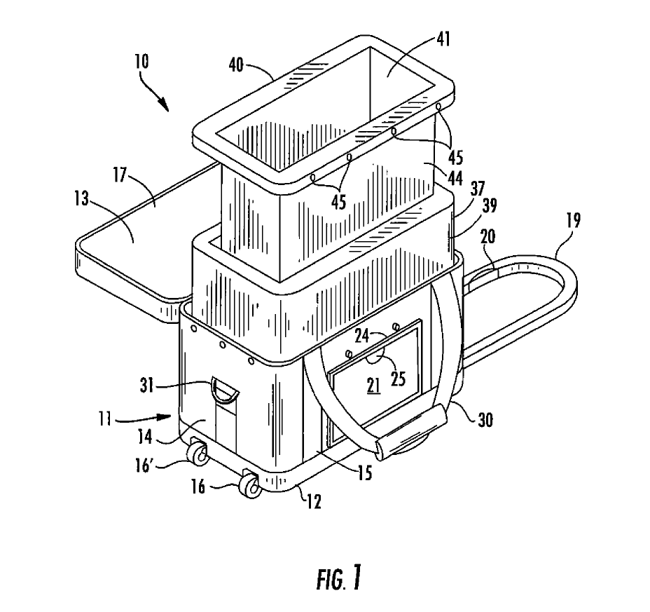

By reference to the exemplary drawings in detail wherein like numerals indicate like elements throughout the various views, there is shown in FIG. 1 an exemplary container 10 for transporting blood and blood products in accordance with the invention.

The container 10 can include an outer case 11 that may be formed from soft, flexible, lightweight material such as ballistic nylon, vinyl or canvas, or a hard rigid material for more durability, or any other suitable materials generally known and used by persons skilled in the art. The outer case 11 is preferably made of waterproof, stain resistant, rustproof material, which can be easily cleaned. The container 10 may be made in different sizes depending on the number of blood or blood product units that are being transported. In one arrangement, the container 10 can hold 1-2 units of blood or blood products. In another arrangement, the container 10 can hold 1-4 units of blood. Alternatively, the container 10 can hold 1-6, or more units of blood or blood products.

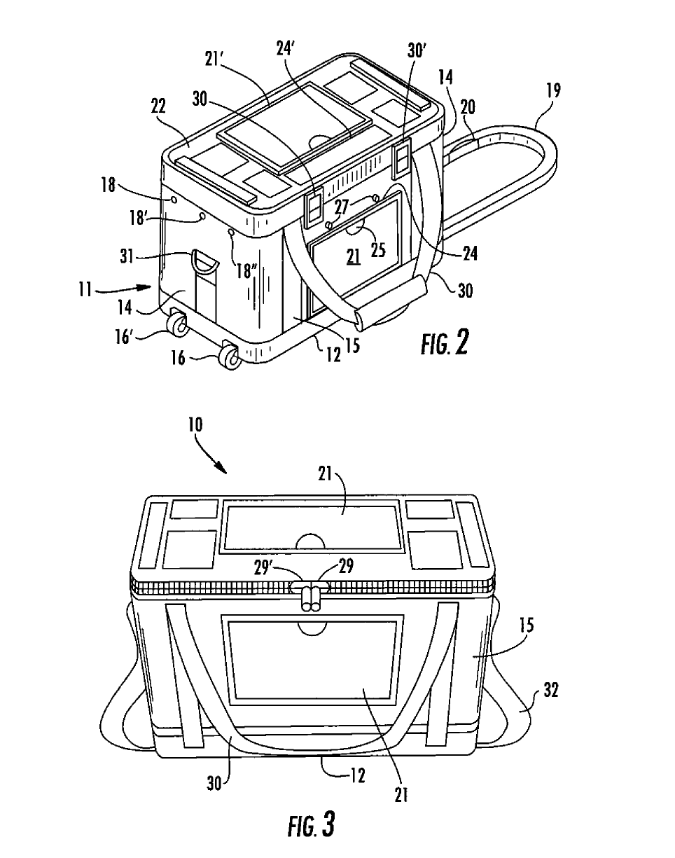

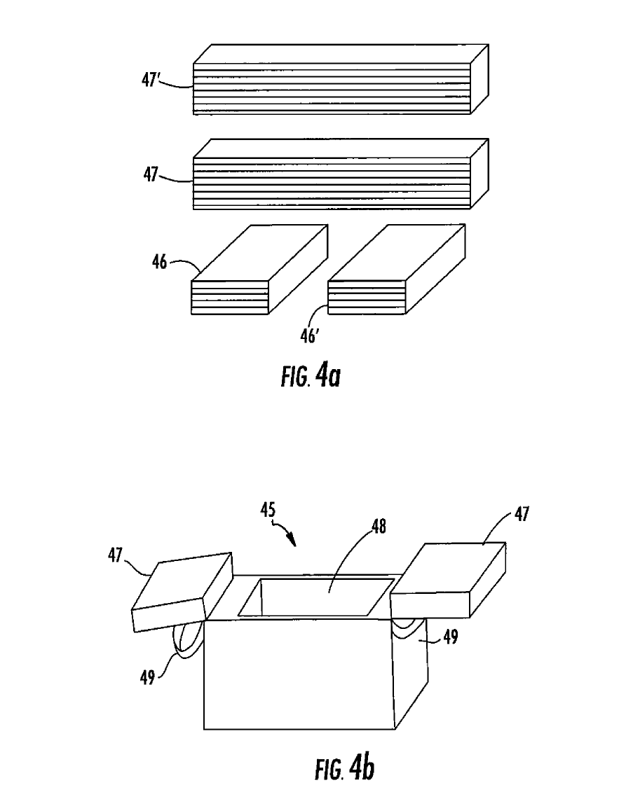

In the arrangement illustrated in FIGS. 1-2, the outer case 11 comprises a base 12 which may be formed from a reinforced or stiff material, or of other suitable materials generally known and used by persons skilled in the art. In an alternate arrangement, illustrated in FIG. 3, the base 12 may include a rigid base insert covered with a flexible material. In the exemplary arrangement shown, the outer flexible material is ballistic nylon, but any suitable material may be employed. The outer case 11 further comprises ends 14 and 14′ that are connected to the base 12, with sides 15 and 15′ (not shown) extending between the ends 14 and 14′.

The container 10 provides for a lid 17, which can be hingedly attached to the side 15 or 15′ (not shown) of the outer case 11 and may be closeable by twist latches 30, 30′. Preferably, at least one twist latch 30 is lockable. Alternatively, other suitable forms of closeable means such as hooks, loops, snap fasteners, buttons, Velcro or zippers may be employed to close the lid 17.

FIG. 3 depicts one arrangement of the invention wherein the lid 17 is hingedly attached to one of the sides 15, and may be closable with at least one zipper 29 and 29′. Alternatively, the zipper 29 may extend around the entire periphery of the lid 17 such that the lid 17 is completely removable. In another arrangement of the invention, the lid 17 may have a zipper 29 extending around two ends 14, 14′ and one (1) side 15 so that the lid 17 may be hinged at its attached side 15 providing maximum opening without being completely removable. However, it will be appreciated by those skilled in the arts of the invention that any form of a lid 17 may be employed.

In one arrangement, the lid's interior 13 comprises closed cell PE foam insert that acts as insulation to prevent the transmittal of external heat, thus protecting the interior contents of the container 10. It will be appreciated that any suitable insulating material may be used in the lid's interior 13. The insulating material 13 may also act as a water barrier to protect the interior contents of the container 10. In another arrangement, a cooling module may be added to the lid's interior 13 in which coolant material may be disposed, further enhancing the performance of the container 11.

As shown in FIG. 2, a wheel assembly 16, 16′ and anchor screws 18, 18′, 18″ located on one of the opposing ends 14 can provide stabilizing means for the container 10 when stood on its end 14. The stabilizing means allow the container 10 to be placed flat on its base 12 without the container 10 tipping over. Other stabilizing means may include at least a foot, claw or any other stabilizing means that is generally known and used by persons skilled in the art. In one arrangement of the invention, a telescopic handle 19 may be provided adjacent to the base 12 for pulling the container 10 on the wheel assembly 16, 16′. The telescopic handle 19 is capable of gradually extending to the desired height from the top end 14′ of the container 10 when a push button 20 is pressed. The push button 20 also serves to retract the telescopic handle 19 when pressed.

The container 10 also preferably comprises of at least one pair of non-retractable handles 30 and 30′ (not shown) providing alternate means for picking up and carrying the container 10. As shown in FIGS. 1 & 2, the outer case 11 provides rings 31 and 31′ (not shown) for a shoulder strap 32 (not shown) to be attached for alternate means for picking up and carrying the container 10. FIG. 3 shows one arrangement of the invention where the shoulder straps are attached to the outer case 11. In certain arrangements of the invention, an additional reinforced handle can be attached to the top of the lid's exterior 22 providing yet another means for picking up and carrying the container

As shown in FIG. 1, the outer case 11 may include at least one identification sleeve 21 which is adapted to secure and hold documentation concerning the blood or blood products being transported, which may include a patient's name, social security or identification number, physician, blood type, date the blood or blood product left the blood bank, scheduled date of delivery, number of units of blood being transported, delivery destination, etc. The identification sleeve 21′ can be made of a clear or transparent plastic with an opening 24 adapted with a finger notch 25 for easy insertion and removal of documentation.

As shown in FIG. 2, in one arrangement of the invention the outer case 11 can have more than one identification sleeve 21, 21′, one on the side 15 of the outer case 11, and the other on the lid's exterior 22. It is understood by those practicing the arts that there can be as many identification sleeves 21, 21′ as desired. As shown in FIG. 2, the identification sleeve 21 may be secured to the side of the container 10 by anchor screws 27, 27′, 27″, 27″, or any other securing means that are generally known and used by those skilled in the pertinent arts, including but not limited to, screws, nails, bolts, paste, glue and Velcro. The lid's exterior 11 is adapted for provision of any desired logo or other identification marks or notations.

In all arrangements, the outer case 11 is adapted to form a receptacle area for an insulating layer 37 made of suitable insulation materials to shield the interior contents of the container 10 from external temperature. Suitable insulation materials may include a substantially rigid insulating material having a relatively low thermal conductivity and being relatively light weight, for example Minicel®, Volara®, Neoprene, Polystyrene (Styrene), Polyethylene, VIP (Vacuum Insulation Panel), ABS and Coroplast®, TempShield™ and SPACE AGE®. Preferably the insulating layer 37 is formed from rigid polyurethane. Alternatively, the insulating layer 37 may be formed from closed cell PE foam, and may be formed of the same material as the lid's interior 13.

The insulating layer 37 includes a bottom portion 38 connected to walls 39 substantially surrounding the inner perimeter of the outer case 11. The insulated layer 37 has a predetermined height to substantially engage or abut the top of the container 10 while substantially minimize the air space remaining above the container 10.

The container 10 also includes a generally rigid waterproof open well 40, which may comprise PE plastic shaped to form a receptacle area 41 for cooling elements 46, 46′, 47, 47′. The insulation deck 40 substantially surrounds the insulated layer 37 and includes a bottom portion 43 connected to walls 44 in a seamless well for spill proofing and to receive the cooling elements 46. After the insulating layer 37 is placed in the outer case 11, the well 40 is inserted in the outer case 11 and substantially engages the walls 39 and bottom portion so as to substantially seal the insulated layer 37 to the outer case 11. In one arrangement of the invention, the well 40 is secured to the outer case 11 by anchor screws 45. It is understood by those skilled in the arts that other securing means such as nails, glue, nuts and bolts may be applied to secure the well 40 to the outer case 11, or that the well 40 may be removable from the outer case 11 for easy cleaning.

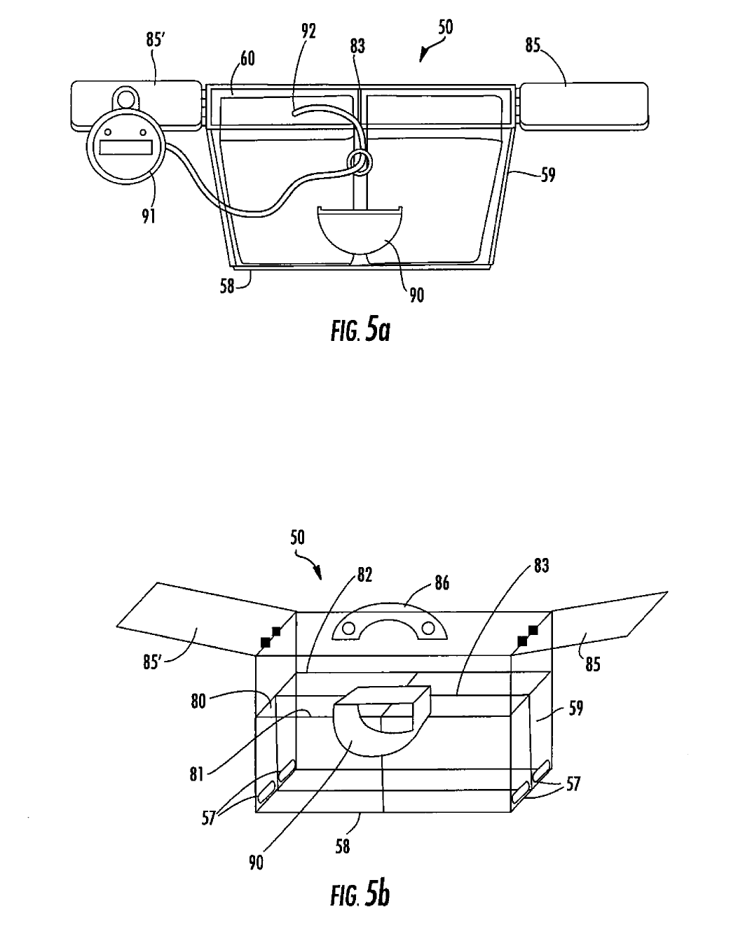

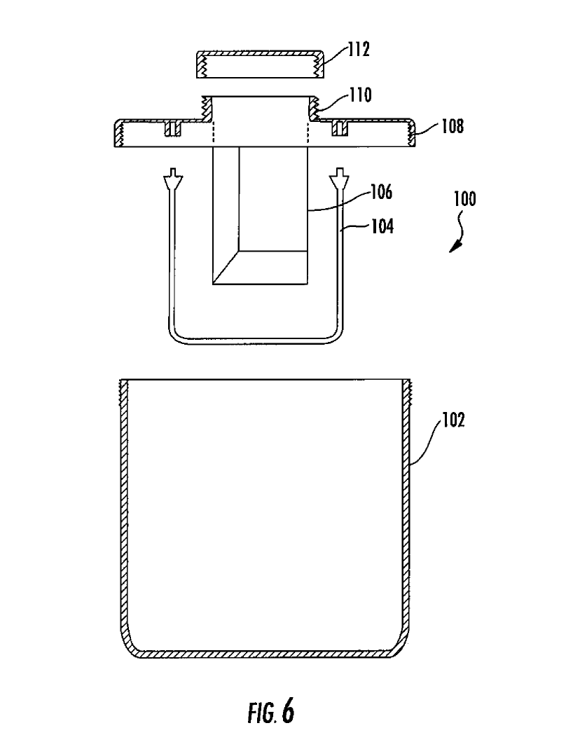

FIG. 4a depicts an arrangement of the cooling element 45 of the present invention, which is responsible for keeping the inner caddy 50 (illustrated in FIG. 5) at the required temperatures of 1° C. to 6° C. The cooling element 45 can be formed of separate components 46, 46′, 47, 47′ that are made from strong, durable and lightweight material and comprise at least four modular units with refrigerant material disposed within each modular unit. The cooling elements 46, 46′, 47, 47′ include two sides 46 and 46′ and a top 47 and bottom 47′. The two sides 46 and 46′ are sized and shaped to securely fit the interior sides of the insulation deck 40, while the top 47 and bottom 47′ are sized and shaped to fit the bottom and top of the well 40.

In another arrangement of cooling element 45, illustrated in FIG. 4b, the cooling element 45 is formed so as to define a receptacle 48 therein, with two hinged top portions 47 attached to side walls 46. The cooling element 45 may also include handles 49 and may be sized to fit within the well 40. The handles 49 enable the cooling element 45 to be easily placed into and out of the well 40. In a further arrangement of cooling element, the top portion 47 may be hinged along a long side thereof. The hinge may be formed of Lucite, fabric, or any suitable material.

The cooling element may be formed of a single material that retains its temperature for a prolonged period of time, or may be formed from a combination of materials. In one arrangement, −1° C. phase change gel packs may be embedded in insulating material, such as Coroplast® or ABS/Starboard plastic (Styrene). 0° C. phase change gel packs may also be used, although such gel packs do not maintain the desired temperature as long as the −1° C. phase change gel packs. The phase change gel packs may be completely sealed within the insulating material, or may be placed in suitable hollows created within the insulating material. When ABS plastic is used, a solvent may be employed to melt and fuse the plastic around the gel pack in order to prevent leakage thereof. By encasing the gel packs in relatively rigid plastic, the gel packs are prevented from bulging due to their contents shifting during thawing and refreezing, which can otherwise encroach on the available storage space for the caddy 50. The cooling element 45 may have two phase change gel packs located in both the top and bottom of the element, and one gel pack in each side.

To use the cooling element 45, the element can be frozen in a commercial or home freezer at temperatures set at or below −10° C. for at least 24 hours, and then conditioned (allowed to stand at room temperature for 30-60 minutes after removal from the freezer) prior to use. If the cooling element 45 is already partially frozen, twelve (12) hours of freezing should prove sufficient. The cooling elements 45 are stackable and easily stored in the freezer. After each use, the cooling element 45 is removed from the container 10 and is refrozen before each day's use. Once the cooling element 45 is frozen and conditioned, it is ready for insertion into the container 10. In the case of separate components, the bottom 47′ is first placed in the bottom of the well 40 and the sides 46 and 46′ are inserted along the sidewall of the well 40 forming a receptacle area 48 for the inner caddy 50. After the inner caddy 50 is loaded and properly secured within the receptacle area 48, then the top 47 is placed on top of the inner caddy 50. In the case of a single cooling element 45, the cooling element is placed in the well 40 by means of the handles 49, and the hinged top portions 47 are opened ready to receive the inner caddy 50.

FIGS. 5a and 5b depict arrangements of the inner caddy 50 made to hold, transport and monitor the temperature of 1 to 6 units of blood or blood products at the required temperature range. Typically, one arrangement of the caddy is sized to receive both 450 cc sized whole blood units, and 350 cc PRBC blood product units. It will be appreciated by those of skill in the art that different caddies may be sized differently for different payloads, as typically stem cells, bone marrow and organs are transported in different sized containers. The appropriately sized caddy should be selected for the item being transported. For example, different caddies to transport 2-6 units of blood, 2-3 units of stem cells or 1-2 units of bone marrow may be sized with the same external dimensions to fit inside a container 10. In this way, it is not necessary to carry a large stock of different sized containers 10 because the inner caddy 50 can be swapped out depending on the particular transport application. The inner caddy 50 is preferably constructed of clear, durable and lightweight material, such as Lucite or polycarbonate material or other like materials that are generally known and used by those skilled in the arts. However, it is understood that any material of like properties that is known and generally used by those skilled in the arts may be employed. The inner caddy 50 is removable and has cleaning vents 57, allowing for self-drainage and easy cleaning. The inner caddy 50 comprises of a bottom portion 58 connected to walls 59.

Preferably, the inner caddy 50 defines a holding area 60 that can accommodate two or more units of blood or blood products at the required temperature range. As best seen in FIG. 5, holding area 60 is defined by sidewalls 80, front wall 81 a back wall 82, and a middle divider 83. To store a unit of blood or blood products in the holding area 60, the unit is inserted in an upright position in between one of the straight side walls 80 and the middle divider 83. The sidewalls 80 and middle divider 83 are sized and shaped to hold and secure a unit of blood or blood products placed between them.

In one arrangement of the invention, the holding area 60 is adapted to hold 1 to 2 units of blood. In another arrangement, the holding area 60 is adapted to hold up to four units of blood. In yet another arrangement, the holding area 60 is adapted to hold six or more units of blood or blood products. The sidewalls 80 are hingedly connected to lids 85, 85′, which either extend outwardly open from the holding area 60 to form gull wings or from front to back (not shown). Each lid 85, 85′ is situated on an opposing side of the inner caddy 50. In one arrangement, lifting means 86 are provided for easy removal of the inner caddy 50 from the container 10. Lifting means may include a handle, strap, tab, or anything that can be grasped or is generally known and used by those skilled in the arts. In one arrangement, lids 85, 85′ include an aperture 87 (not shown) adapted to receive the lifting means. The inner caddy may be kept refrigerated with the blood or blood products stored therein prior to use for transportation purposes.

In one arrangement of the invention, the lids 85, 85′ are adapted to receive tamper resistant security tags or clips. If the tamper resistant security tags are still in place at the destination address for the blood or blood products being transported, then the recipient can be assured that the blood or blood products was not removed from the container 10. The tamper resistant security tags provide an effective means of ensuring compliance with certain protocols.

As shown in FIGS. 5a and 5b, the inner caddy 50 provides for a receiving area 90, which is sufficiently wide and deep enough to hold and secure a temperature data-logging device 91. In the preferred arrangement, the receiving area 90 is secured to the front wall 81 of the inner caddy 50. However, it is appreciated by those skilled in the arts that the receiving area 90 may be located anywhere on or near the inner caddy provided that the temperature data-logging device 91 is proximately located near the blood or blood units being transported for monitoring purposes.

The temperature data-logging device 91 comprises of at least one probe 92 for monitoring, collecting and reporting data concerning the blood or blood units being transported. The probe 92 may be inserted into a protected channel formed in the caddy 50, proximate to the units of blood or blood products being transported. The temperature data-logging device 91 is pre-programmable and can be adapted to monitor the temperature of the blood or blood products being transported for 24+ hours to meet the courier or recipient's protocols. In this manner, compliance and performance of the invention can be documented and verified. The temperature data-logging device 91 may include a connection point to enable it to be connected to a computer docking station for downloading the temperature data, or may connect with a computer wirelessly. Typically, the temperature data is downloaded into suitable software that can produce a graph showing the trend in temperature recorded by the probe 92 over time.

After the cooling element 45 has been frozen, conditioned and placed in the container 10, the inner caddy 50 is removed from the refrigerator with the units of blood or blood products and inserted into the receptacle area 48 formed by the cooling element 45 for receiving the inner caddy 50. The temperature data-logging device 91 is now ready for monitoring and can be positioned in the receiving area 90 with its probe inserted in the holding area 60 at or near the units being transported. The temperature data-logging device 91 may be activated by swiping it with a start magnet, or otherwise switched on. Thereafter, the temperature data-logging device 91 can continuously monitor the units of blood or blood products for up to 24+ hours. The temperature data-logging device 91 can be programmed to either transmit an alarm or a page if the temperature range is outside the set parameters. Alternatively, or in addition, a user can download data from the data-logging device 91 once the container 10 has reached its final destination, and can view the temperature information on a computer or can print out the information.

The container 10 may also be equipped with a cell phone/GPS unit such that a user can call the cell phone in order to find out the location, speed of travel, temperature, etc of the unit. It will be appreciated that any suitable communications device may be used, and that a cell phone is used as an example only. It is possible to program geographical parameters outside which the container 10 should not travel, and have the cell phone call or page a user if the container 10 is found to be outside this area. This can help to alleviate routing errors in transportation which cause the blood or blood products to be in transit for longer than is desirable. The cell phone/GPS unit may be incorporated into the temperature data-logging device 91, or may be separate therefrom. Preferably, the temperature data-logging device 91 sends temperature data to the cell phone/GPS unit so that the temperature information may be transmitted by the cell phone. Preferably, the communication between the data-logging device 91 is carried out wirelessly. The antenna for the cell phone/GPS unit functions optimally when the exterior of the case 11 is formed of ballistic nylon or other suitable material that does not interfere with reception. Batteries for the cell phone/GPS unit are also included. Additionally, sensors may be included for monitoring opening of the container 10, and/or the caddy 50. A processor may be included.

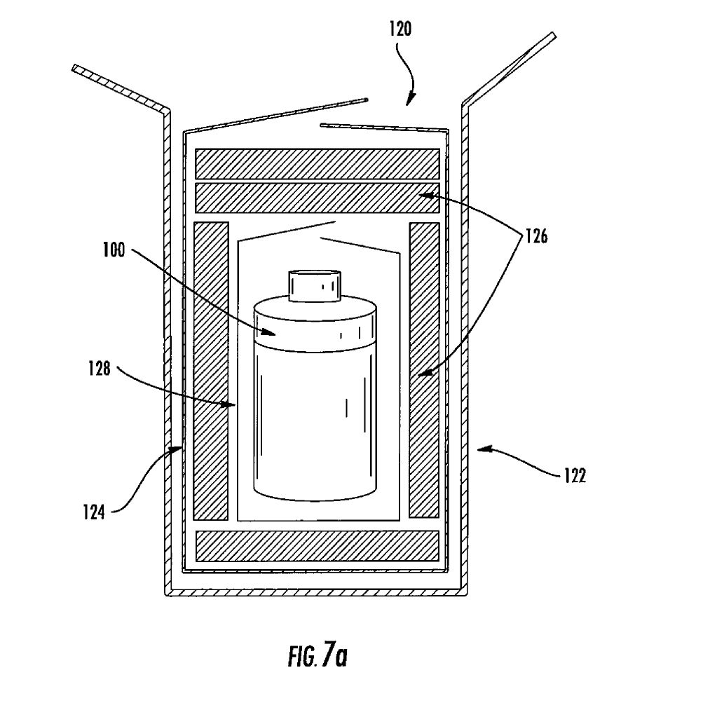

Another arrangement of the invention is illustrated in FIG. 6 for transporting test tubes and vials of specimens. The container 100 of FIG. 6 is smaller than the container of FIGS. 1-5, because it is sized to receive tubes or vials of specimens rather than bags of blood or blood products. The container 100 may comprise an outer plastic layer 102, a inner insulating layer 104, and an inner caddy 106. The layers 102, 104 and 106 may be engaged with an end cap 108, which may have screw threading 110 thereon to engage with a cap 112. Preferably all the components are formed of plastic or other non-reactive material so that any leakage of a phase change gel incorporated between the insulating 104 and the outer layer 102 or the inner caddy 106 will not react with the container 100. It is also preferred that all the components are sealed together. The entire container 100 may be frozen for 24 hours prior to use. The test tubes or vials to be transported are wrapped in a plastic, polyethylene or other suitable bag, or in bubble wrap, and are placed within the inner caddy 110.

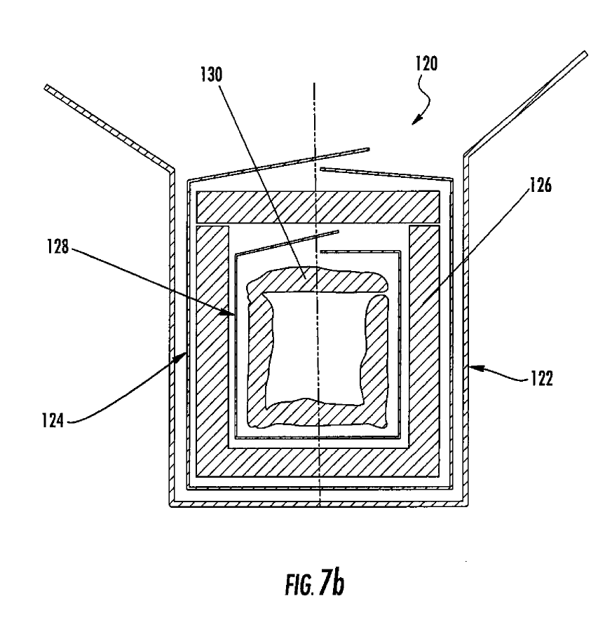

A further arrangement of the invention is illustrated in FIGS. 7a and 7b. In this arrangement, the container 120 comprises an outer corrugated layer 122, a large flexible bag 124, which may be formed of Mylar or any suitable material, an insulating layer 126 of open cell or closed cell foam, or any other suitable material, a second smaller Mylar or other flexible bag 128. The Mylar or other foil bags provide air barriers to aid with insulation of the product contained within the second flexible bag 128. The bags 124 and 128 may be open at the top, and may be simply closed in an overlapping manner using a gift warp type fold, meaning that a user does not need to use glue or tape to keep the bags closed. The insulating layer 126 may be formed as six separate pieces to surround the inner compartment, or may include fewer pieces that are folded or otherwise joined. Preferably at least the top of the insulating layer is removable from or hingedly attached to the walls so that access to the interior of the container 120 may be easily gained. The product to be transported may be placed directly inside the compartment created by the bag 128, or may be placed within cooling gel packs (not shown) placed in the bag 128. The product may be placed inside a material such as bubble wrap prior to being placed inside the container 120, in order to prevent any cold spots forming in the product in any location where the product may directly touch the gel packs. The container 120 can transport products for a minimum of 48-96 hours at the required temperature. Larger containers 120 may be employed to transport pallets of products, for example for military or laboratory use. The container 100 of FIG. 6 may be used within the container 120 to provide additional temperature stability to the container 100, as shown in FIG. 7a, without the use of gel packs.

As shown in FIG. 7b, the container 120 can be used to transport products directly, using gel packs 130. In the illustrated arrangement, six −1° C. gel packs 130 are employed, which may have different sizes such that 2 larger packs may go on the top and bottom of a specimen to be transported, with 4 smaller packs around the sides of the specimen. The 4 smaller packs may be connected together to form a single wall that may be folded easily at the joins between the packs. Different phase change gel packs 130 may be switched in and out of the container 120, depending on the type of specimen that is to be transported. For example, −18° C. to −25° C. phase gels or +2° C. to +5° C. phase gels may be used, depending on the temperature at which the product is to be transported. The gel packs are frozen prior to use, preferably for at least 24 hours, and the product to be transported is surrounded by the frozen packs. Alternatively, phase gel packs incorporated into cooling elements 45 may be used, with the cooling elements sized depending on the product that is to be transported.

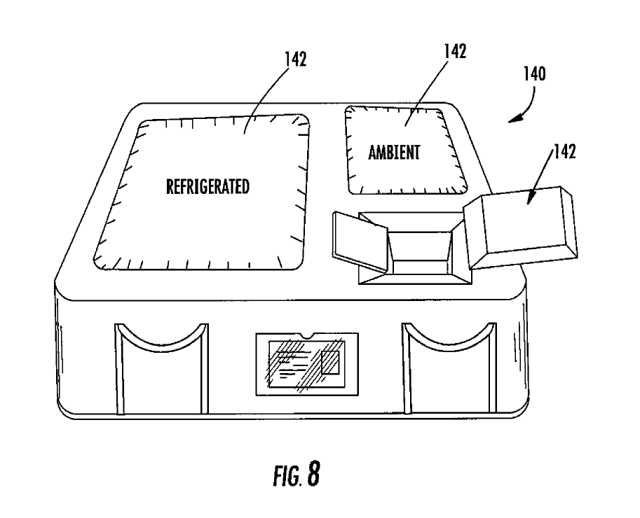

In another arrangement of the invention, illustrated in FIG. 8, different temperature zones may be created within the same container 140 for the transport of different products at the same time. For example, three different temperature zones such as ambient, refrigerated and frozen may be included. In order to do this, the insulating layers 142 may be arranged to create compartments of different sizes. The container 100 of FIG. 6 may be used within the container 140 to create a frozen temperature zone. The temperature zones may also be used to control the length of time that the container 140 may be used, such that the container 140 may be used for 12, 24, 48 or 72 hours depending on the initial temperature of the phase change gel packs.

All the arrangements of the invention result in lightweight, reusable shipping containers for blood, blood products and other biological specimens and products, and which keep the temperature of the products within the FDA required time periods. Monitoring of the temperature may be used with any of the arrangements, by using a temperature data-logging device 91 inside any of the containers.

Further modifications and alternative arrangements of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred arrangements. Elements and materials may be substituted for those illustrated and described herein, parts and processes may be reversed, and certain features of the invention may be utilized independently, all as would be apparent to one skilled in the art after having the benefit of this description of the invention. Changes may be made in the elements and compositions described herein or in the features or in the sequence of features of the methods described herein without departing from the spirit and scope of the invention as described in the following claims.

What is claimed is:

1. A container for transporting blood and blood products comprising:
an outer case including a lid;
an insulating layer substantially inside the outer case and the lid;
a generally rigid water-resistant well having a lip, wherein the lip abuts walls of the outer case so as to substantially seal the insulated layer to the outer case;
at least one cooling element, the at least one cooling element being capable of maintaining temperature within a prescribed temperature range for predetermined periods of time upon being kept at a predetermined temperature prior to use, the at least one cooling element being disposed within the well and at least partially surrounding a receptacle area; and
a caddy located in said receptacle area and designed to accommodate at least one unit of blood or blood products, wherein said caddy is at least partially surrounded by the at least one cooling element.

2. The container of claim 1, further comprising a temperature data-logging device for monitoring, collecting and reporting data concerning the blood and blood products being transported.

3. The container of claim 1, wherein the cooling element comprises cooling material disposed therein, the cooling material comprising a phase change gel.

4. The container of claim 3, wherein the phase change gel is sealed within the cooling element.

5. The container of claim 1, wherein the cooling element at least partially comprises an ABS plastic.

6. The container of claim 1, wherein the cooling element is shaped so as to substantially surround the caddy.

7. The container of claim 6, wherein the cooling element comprises a bottom, side walls and at least one top portion, wherein the top portion is hingedly attached to the side walls.

8. The container of claim 1, wherein the cooling element is removable from the container so as to be separately freezable prior to use.

9. The container of claim 1, wherein the outer case further comprises means for lifting and towing the container.

10. The container of claim 9, wherein the lifting and towing means further comprises a reinforced handle.

11. The container of claim 9, wherein the lifting and towing means further comprises a wheel assembly.

12. The container of claim 9, wherein the lifting and towing means further comprises of a telescopic handle.

13. The container of claim 9, wherein the lifting and towing means further comprises of a non-retractable handle.

14. The container of claim 9, wherein the lifting and towing means further comprises of a shoulder strap.

15. The container of claim 1, wherein the outer case further comprises of at least one information sleeve adapted to hold identification information concerning the blood or blood product being transported.

16. The container of claim 1, wherein the lid is hingedly attached to the outer case and includes closing means for closing the container.

17. The container of claim 16, wherein the closeable means comprises twist latches.

18. The container of claim 16, wherein the closing means comprises at least one zipper.

19. The container of claim 1, wherein the insulating layer is formed of polyurethane material.

20. The container of claim 1, wherein the well is formed of PE plastic.

21. The container of claim 1, wherein the lifting means of the inner caddy includes a handle.

22. The container of claim 1, further comprising a GPS device for tracking the position of the container during transport.

23. The container of claim 23, further comprising a communications device capable of communicating at least one of temperature and position data to a user.

24. A container for transporting blood and blood products comprising:
an outer case including a lid;
an insulating layer substantially inside the outer case and the lid; and
at least two cooling elements, the cooling elements each being capable of maintaining temperature within a prescribed temperature range for predetermined periods of time upon being kept at a predetermined temperature prior to use, the cooling elements being disposed within the insulating layer and each defining a receptacle area designed to accommodate at least one unit of blood or blood products,
wherein each cooling element is designed to maintain blood or blood products located within the receptacle area at a different predetermined temperature.

26. The container of claim 25, wherein each cooling element includes phase change gel.

27. The container of claim 25, further comprising a temperature data-logging device for monitoring, collecting and reporting data concerning the blood and blood products being transported.

28. A container for transporting blood and blood products comprising:
an outer case including a lid;
an insulating layer substantially inside the outer case and the lid;
a flexible foil barrier positioned between the outer case and the insulating layer;
at least one cooling element, the at least one cooling element being capable of maintaining temperature within a prescribed temperature range for predetermined periods of time upon being kept at a predetermined temperature prior to use, the at least one cooling element being disposed within the flexible foil barrier and at least partially surrounding a receptacle area; and
a receptacle located in said receptacle area and designed to accommodate at least one unit of blood or blood products, wherein said receptacle is at least partially surrounded by the at least one cooling element.

29. The container of claim 28, further comprising a second flexible foil barrier surrounding said receptacle.

30. The container of claim 28, wherein the at least one cooling element includes phase change gel.

31. The container of claim 30, wherein the phase change gel is sealed within sealed within the cooling element.

32. The container of claim 30, wherein the cooling element at least partially comprises an ABS plastic.
